(19) United States
(12) Patent Application Publication
(10) Pub. No.: US 2011/0040350 A1
(43) Pub. Date: Feb. 17, 2011

(54) FSK telemetry for cochlear implant

(76) Inventor: Glen A. Griffith, Newbury Park, CA (US)

Correspondence Address: Wong Cabello Lutsch Rutherford & Brucculeri LLP, 20333 Tomball Pkwy, Suite 600, Houston, TX, 77070, US

(21) Appl. No.: 11/122,648

(22) Filed: May 5, 2005

(51) Int. Cl. A61F 11/04; A61N 1/36

(52) U.S. Cl. 607/57

(57) ABSTRACT

An apparatus for stimulating a sensory organ includes an external portion and an internal portion. The external portion is configured for wireless transmission of an FSK signal having encoded therein information indicative of sensory stimuli. The internal portion, which is in data communication with the external portion, is configured for wireless reception of the FSK signal and for causing stimulation of the sensory organ in response to the information encoded in the FSK signal.

### TECHNICAL FIELD OF DISCLOSURE

This disclosure is directed to cochlear implants, and in particular, to the transmission of data between external and internal portions of an implant.

### BACKGROUND

Perception of sound begins when a sound wave strikes the eardrum, thereby causing it to vibrate. Vibration of the eardrum in turn causes vibration of small bones in the middle-ear, to which the eardrum is mechanically coupled. These bones transmit the energy from the sound wave into a fluid that fills the cochlea, thereby initiating a pressure wave that propagates through the fluid.

The pressure wave brushes past hairs that line the interior of the cochlea, setting those hairs into motion as it does so. These hairs are coupled to auditory nerves. Hence, stimulation of the hairs results in nerve stimulation. The extent to which the hairs are bent determines the loudness of the sound. The location of the hair within the cochlea determines the frequency, or pitch of the sound.

In certain diseases, the cochlea develops what amounts to bald spots. These bald spots result in loss of the ability to perceive those frequencies that correspond to the locations of those bald spots. Cochlear implants provide electrodes that mimic the function of those missing hairs by applying electric fields to stimulate selected portions of the cochlea in response to detected sound.

A cochlear implant has an associated external microphone and signal processing system to generate a sensory signal that provides information for controlling the application of local electric fields by the implant. This sensory signal is then provided to the cochlear implant.

Because of constraints imposed by the anatomy of the ear, it is generally considered impractical to connect the signal processing system to the cochlear implant by a wire. The conventional approach is to modulate an RF carrier frequency with the sensory signal to generate a modulated RF signal. This modulated RF signal is then sent transcutaneously to an implanted receiver.

A difficulty with this approach is that there is a great deal of information in the sensory signal. As a result, the modulated RF signal has a large bandwidth. For example, in a typical application, the data rate associated with the sensory signal is on the order of 500 kilobytes per second to 1 megabyte per second. This results in a bandwidth of 3-5 megahertz surrounding the carrier frequency. This results in electromagnetic interference that is sufficient to render such systems non-compliant with various international standards.

### SUMMARY

The systems and techniques described herein provide ways to reduce electromagnetic interference resulting from transcutaneous telemetry.

In one aspect, the invention includes an apparatus for stimulating a sensory organ. The apparatus includes an external portion and an internal portion. The external portion is configured for wireless transmission of an FSK signal having encoded therein information indicative of sensory stimuli. The internal portion, which is in data communication with the external portion, is configured for wireless reception of the FSK signal and for causing stimulation of the sensory organ in response to the information encoded in the FSK signal.

Embodiments of the invention include those in which the external portion is configured to transmit a 16-ary FSK signal, as well as those in which the external portion is configured to generate an FSK signal by direct digital synthesis.

In some embodiments, the external portion includes a memory having stored therein samples of a first waveform, and an accumulator having stored therein information for retrieving selected samples of the first waveform.

In other embodiments, the receiver includes a phase-locked loop for generating a control signal representative of a frequency of the FSK signal.

Additional embodiments include those in which the external portion is configured to encode information representative of ambient sound in the FSK signal, and the internal portion is configured to cause stimulation of a cochlea on the basis of the encoded information.

The internal portion can include an array of electrodes disposed to stimulate different portions of the cochlea in response to the encoded information.

In another aspect, the invention includes a cochlear implant system having an externally-worn portion and an implantable portion. The externally-worn portion includes a transmitter configured for wireless transmission of an FSK signal having encoded therein information indicative of audio stimuli. The implantable portion includes a receiver configured for wireless reception of the FSK signal. A processor generates stimulus signals on the basis of the received FSK signal and provides those signals to an an electrode array responsive to the stimulus signals.

In another aspect, the invention is a method for stimulating a sensory organ. Such a method includes generating an FSK signal having encoded therein information representative of ambient stimuli; wirelessly transmitting the FSK signal transcutaneously to an implanted receiver; and stimulating the sensory organ in response to the information encoded in the FSK signal.

Certain practices of the invention includes those in which generating an FSK signal includes generating a 16-ary FSK signal.

Other practices include those in which generating an FSK signal includes sampling, at selected intervals, a first waveform having a first frequency, thereby generating a second waveform having a second frequency, the second frequency depending on the selected intervals.

In some practices of the invention, an FSK signal is provided to a phase-locked loop. Information encoded in the FSK signal is then encoded into a voltage-controlled oscillator control signal generated by the phase-locked loop.

Optionally, the method includes selecting the ambient stimuli to be ambient sound, and wherein stimulating the sensory organ includes stimulating a cochlea.

Some practices of the invention also include stimulating the cochlea by selectively exciting electrodes disposed along the cochlea.

These and other features and advantages will be apparent from the following detailed description and the accompanying figures, in which:

### BRIEF DESCRIPTION OF THE DRAWINGS

FIG. 1 shows a representative stimulation pulse;

FIG. 2 shows a representative cochlear stimulation system for delivering the stimulation pulse of FIG. 1;

FIG. 3 is a block diagram of a speech processor used in the system of FIG. 2A;

FIGS. 4 and 5 show portions of an electrode array deployed in the cochlea;

FIG. 6 is a block diagram of a transmitter for transmitting an FSK modulated telemetry signal to an implanted receiver, or “implant;”

FIGS. 7 and 8 are block diagrams of transmitters that use direct digital synthesis to generate telemetry signals; and

FIG. 9 is a block diagram of a receiver for receiving an FSK modulated telemetry signal.

### DETAILED DESCRIPTION

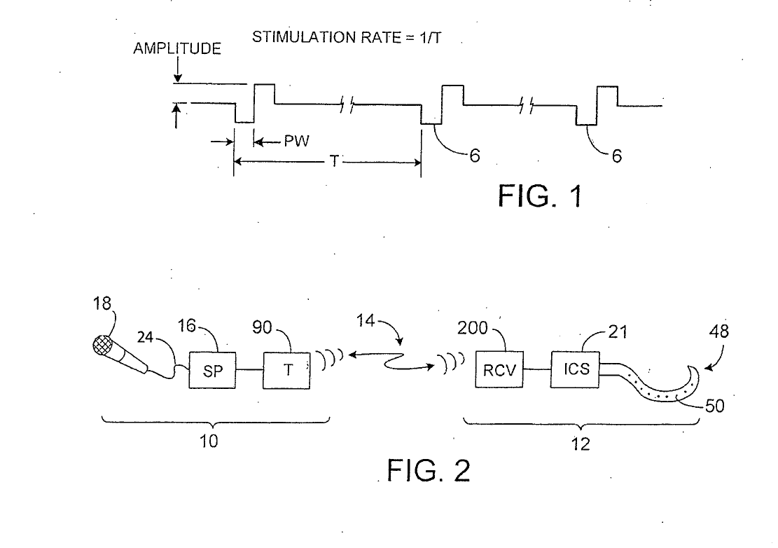

FIG. 1 shows a biphasic pulse train having a stimulation rate (1/T), pulse width and pulse amplitude as those terms are commonly used in connection with a neurostimulator device, such as a cochlear implant, a spinal cord stimulator, a deep brain stimulator, or other neural stimulator. All such systems commonly stimulate tissue with biphasic pulses 6 of the type shown in FIG. 1.

A “biphasic” pulse 6 consists of two pulses: a first pulse of one polarity having a specified magnitude, followed immediately, or shortly thereafter, by a second pulse of the opposite polarity, although possibly of different duration and amplitude. The amplitudes and durations are selected so that the total charge of the first pulse equals the total charge of the second pulse. Such charge-balancing is believed to reduce damage to stimulated tissue and to reduce electrode corrosion. For multi-channel cochlear stimulators, it is common to apply a high rate biphasic stimulation pulse train to each of the pairs of electrodes in the implant (described below) in accordance with a selected strategy and to modulate the pulse amplitude of the pulse train as a function of information contained within a feedback acoustic signal.

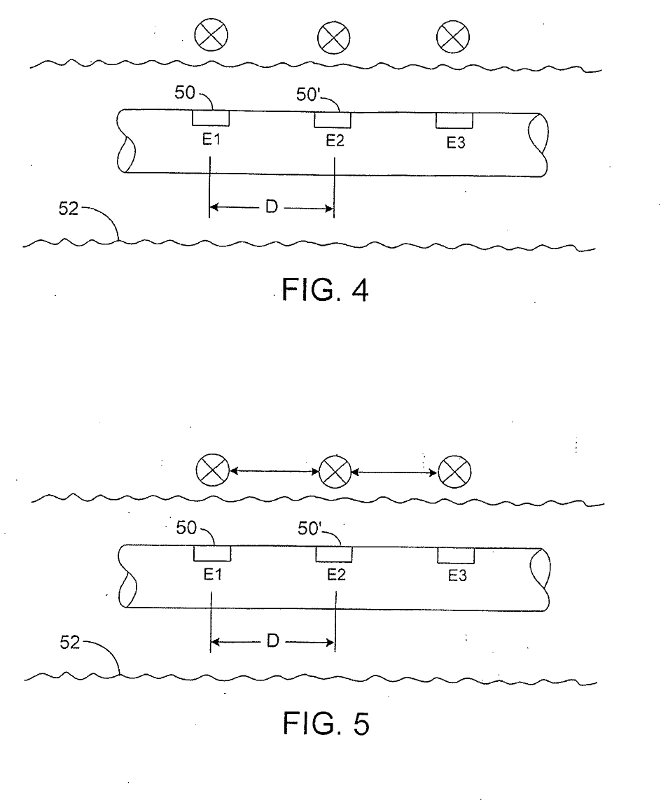

A cochlear stimulation system 5, as shown in FIG. 2, includes a speech processor portion 10 and a cochlear stimulation portion 12. The speech-processor portion 10 includes a microphone 18, a speech processor 16, and a transmitter 90. The microphone 18 may be connected directly to the speech processor 16 or coupled to the speech processor 16 through an appropriate communication link 24 as described herein. The transmitter 90 is described in more detail in connection with FIGS. 4-6.

The cochlear-stimulation portion 12 includes a receiver 200, described below in connection with FIG. 7, an implantable cochlear-stimulator 21, and an electrode array 48 adapted for insertion within the cochlea of a patient. The array 48 includes a plurality of electrodes 50 spaced along the array length. These electrodes 50 are selectively connected to the implantable cochlear-stimulator 21. In typical embodiments, there are sixteen electrodes 50, however there exist embodiments with as few as four to as many as sixty-four electrodes 50. Each electrode 50 in the array is a platinum-iridium electrode.

Typical electrode arrays 48 include those described in U.S. Pat. Nos. 4,819,647 or 6,129,753, both of which are incorporated herein by reference. Electronic circuitry within the implantable cochlear-stimulator 21 allows a specified stimulation current to be applied to selected pairs, or groups, of the individual electrodes 50 within the electrode array 48 in accordance with a specified stimulation pattern defined by the speech processor 16.

The implantable cochlear-stimulator 21 and the speech processor 16 are linked by a suitable data or communications link 14. In some cochlear implant systems, the speech processor 16 and microphone 18 comprise the external portion of the cochlear implant system and the implantable cochlear-stimulator 21 and electrode array 48 comprise the implantable portion of the system. In such cases, the data link 14 is a transcutaneous data link that allows power and control signals to be sent from the speech processor 16 to the implantable cochlear-stimulator 21. In some embodiments, data and status signals may also be sent from the implantable cochlear-stimulator 21 to the speech processor 16.

Certain portions of the cochlear stimulation system 5 can be contained in a behind-the-ear unit that is positioned at or near the patient's ear. For example, the behind-the-ear unit can include the speech processor 16 and a battery module, both of which are coupled to a corresponding implantable cochlear-stimulator 21 and an electrode array 48. A pair of behind-the-ear units and corresponding implants can be communicatively linked via a Bionet System and synchronized to enable bilateral speech information conveyed to the brain via both the right and left auditory nerve pathways. The Bionet system uses an adapter module that allows two behind-the-ear units to be synchronized both temporally and tonotopically to maximize a patient's listening experience.

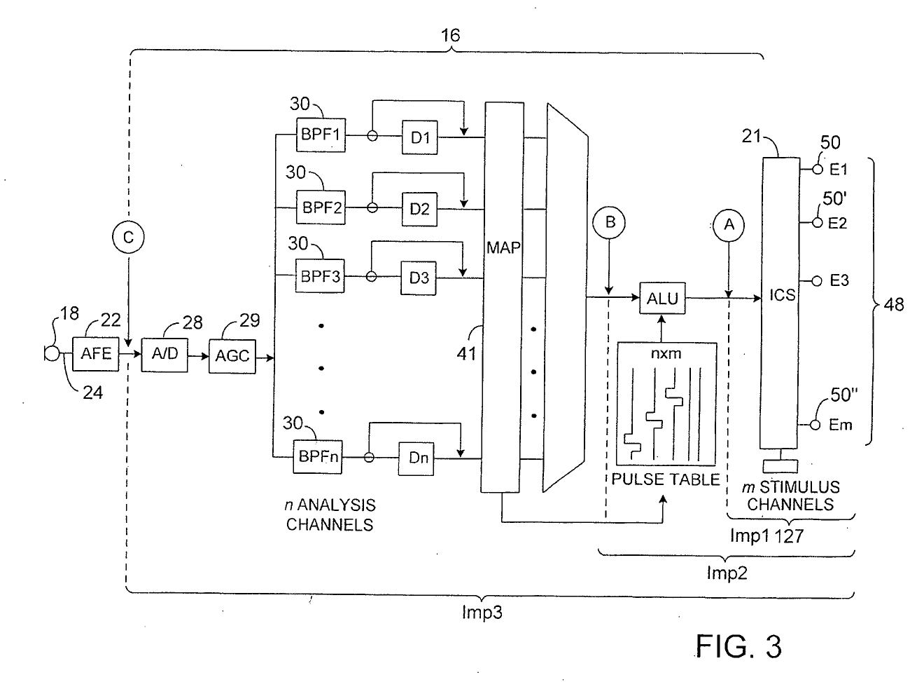

FIG. 3 shows a partial block diagram of one embodiment of a cochlear implant system capable of providing a high pulsatile stimulation pattern to virtual electrodes by appropriately weighting stimuli applied to real electrodes 50. At least certain portions of the speech processor 16 can be included within the implantable portion of the overall cochlear implant system, while other portions of the speech processor 16 can remain in the external portion of the system. In general, at least the microphone 18 and associated analog-front-end (“AFE”) circuitry 22 can be part of the external portion of the system and at least the implantable cochlear-stimulator 21 and electrode array 48 can be part of the implantable portion of the system. As used herein, the term “external” means not implanted under the skin or residing within the inner ear. However, the term “external” can also mean residing within the outer ear, residing within the ear canal or being located within the middle ear.

Typically, a transcutaneous data link between the external portion and implantable portions of the system is implemented by using an internal antenna coil within the implantable portion, and an external antenna coil within the external portion. In operation, the external antenna coil is aligned over the location at which the internal antenna coil is implanted, thereby inductively coupling the coils to each other. This allows data (e.g., the magnitude and polarity of a sensed acoustic signals) and power to be transmitted from the external portion to the implantable portion.

A wireless link between the speech processor 16 and stimulator 21 is described, in U.S. Pat. No. 6,067,474, the contents of which are herein incorporated by reference. The link 14 may be an inductive link using a coil or a wire loop coupled to the respective parts.

The microphone 18 converts incident sound waves into corresponding electrical signals. The electrical signals are sent to the speech processor 16 over a suitable electrical or other link 24. The speech processor 16 processes these signals in accordance with a selected speech processing strategy to generate appropriate control signals for controlling the implantable cochlear-stimulator 21. Such control signals specify the polarity, magnitude, which electrode pair or electrode group is to receive the stimulation current, and when each electrode pair is to be stimulated. Such control signals thus combine to produce a desired time-varying electric field distribution in accordance with a desired speech processing strategy.

A speech processing strategy conditions the magnitude and polarity of the stimulation current applied to the implanted electrodes of the electrode array 48. Such a speech processing strategy involves defining a pattern of stimulation waveforms that are to be applied to the electrodes as controlled electrical currents.

FIG. 3 depicts the functions that are carried out by the speech processor 16 and the implantable cochlear-stimulator 21. It should be appreciated that the functions shown in FIG. 3 (dividing the incoming signal into frequency bands and independently processing each band) are representative of just one type of signal processing strategy that may be employed. Other signal processing strategies could just as easily be used to process the incoming acoustical signal. A description of the functional block diagram of the cochlear implant shown in FIG. 3 is found in U.S. Pat. No. 6,219,580, the contents of which are incorporated herein by reference. The system and method described herein may be used with cochlear systems other than the system shown in FIG. 3.

The cochlear implant functionally shown in FIG. 3 provides n analysis channels that may be mapped to one or more stimulus channels. That is, after the incoming sound signal is received through the microphone 18 and the analog front end circuitry (AFE) 22, the signal can be digitized in an analog-to-digital (A/D) converter 28 and then subjected to appropriate gain control (which may include compression) in an automatic gain control (AGC) unit 29.

After appropriate gain control, the signal can be divided into n analysis channels 30, each of which includes at least one bandpass filter, BPFn, centered at a selected frequency. The signal present in each analysis channel 30 is processed as described more fully in the U.S. Pat. No. 6,219,580 patent, or as is appropriate, using other signal processing techniques. Signals from each analysis channel may then be mapped, using a mapping function 41, so that an appropriate stimulus current of a desired amplitude, polarity, and timing may be applied through a selected stimulus channel to stimulate the auditory nerve.

The exemplary system of FIG. 3 provides n analysis channels for analysis of an incoming signal. The information contained in these n analysis channels is then appropriately processed, compressed and mapped to control the actual stimulus patterns that are applied to the user by the implantable cochlear-stimulator 21 and its associated electrode array 48.

The electrode array 48 includes a plurality of electrode contacts 50, 50′50″ and labeled as E1, E2, . . . Em, respectively, which are connected through appropriate conductors to respective current generators or pulse generators within the implantable cochlear-stimulator. These electrode contacts define m stimulus channels 127 through which individual electrical stimuli can be applied at m different stimulation sites within the patient's cochlea or other tissue stimulation site.

It is common to use a one-to-one mapping scheme between the n analysis channels and the m stimulus channels 127 that are directly linked to m electrodes 50, 50′, 50″, such that n analysis channels=m electrodes. In such a case, the signal resulting from analysis in the first analysis channel may be mapped, using appropriate mapping circuitry 41 or equivalent, to the first stimulation channel via a first map link, resulting in a first cochlear stimulation site (or first electrode). Similarly, the signal resulting from analysis in the second analysis channel of the speech processor may be mapped to a second stimulation channel via a second map link, resulting in a second cochlear stimulation site, and so on.

In some instances, a different mapping scheme may prove beneficial. For example, assume that n is not equal to m (n, for example, could be at least 20 or as high as 32, while m may be no greater than sixteen, e.g., 8 to 16). The signal resulting from analysis in the first analysis channel may be mapped, using appropriate mapping circuitry 41 or equivalent, to the first stimulation channel via a first map link. This results in a first stimulation site (or first area of neural excitation). Similarly, the signal resulting from analysis in the second analysis channel of the speech processor may be mapped to the second stimulation channel via a second map link. This results in a second stimulation site. Also, the signal resulting from analysis in the second analysis channel may be jointly mapped to the first and second stimulation channels via a joint map link. This joint link results in a stimulation site that is somewhere in between the first and second stimulation sites.

The “in-between” site at which a stimulus is applied may be viewed as a “stimulation site” produced by a virtual electrode. Advantageously, this capability of using different mapping schemes between n speech processor analysis channels and m implantable cochlear-stimulator stimulation channels to thereby produce stimulation sites corresponding to virtual electrodes provides a great deal of flexibility in positioning the neural excitation areas precisely in the cochlea.

As explained in more detail below in connection with FIGS. 4 and 5, through appropriate weighting and sharing of currents between two or more physical electrodes, it is possible to provide a large number of virtual electrodes between physical electrodes, thereby effectively steering the location at which a stimulus is applied to almost any location along the length of the electrode array.

An exemplary output stage of the implantable cochlear-stimulator 21, which connects with each electrode E1, E2, E3, . . . Em of the electrode array, is described in U.S. Pat. No. 6,181,969, the contents of which are incorporated herein by reference. Such an output stage advantageously provides a programmable N-DAC or P-DAC (where DAC stands for digital-to-analog converter) connected to each electrode so that a programmed current may be sourced to or sunk from the electrode. Such a configuration permits pairing any electrode with any other electrode and adjusting the complex amplitudes of the currents to gradually shift the stimulating current that flows from one electrode, through the tissue, to another adjacent electrode or electrodes. This enables one to gradually shift the current from one or more electrodes to another electrode(s). Through such current shifting, the stimulus current may be shifted or directed so that it appears to the tissue that the current is coming from or going to an almost infinite number of locations.

The data link 14 between the speech processor 16 and the ICS 21 is a wireless data link in which a transmitter 90, associated with the speech processor 16, modulates a sensory signal representative of ambient sound onto an RF carrier. The RF carrier frequency is one that is allocated for medical use, such as the ISM (Instrument, Scientific, and Medical) frequency of 27.12 megahertz.

An RF carrier is conveniently represented as an exponential function having a complex argument. For this reason, the exponential function is often called a “complex exponential” function. The argument of this complex exponential function has two terms: a frequency term that governs the frequency of the RF carrier and a phase term that governs the phase offset of the carrier.

One can encode information on the carrier by modulating either one of these terms. In “frequency modulation,” it is the frequency term that is modulated. In “phase modulation,” it is the phase term that is modulated.

When encoding digital information on the carrier, it is useful to exploit the fact that a digital signal is only permitted to take on a certain number of values. These allowed values are called “symbols.” The set of all possible symbols is called an “alphabet.”

One way to encode digital information on a carrier is to assign a particular value of the complex argument to each such symbol. This particular type of modulation is called either “frequency shift keying” or “phase shift keying” depending on whether a frequency or a phase is being assigned to a symbol.

Shift keying methods differ in the number of symbols in the alphabet. In general, the fewer the symbols, the easier it is to tell them apart. Thus, with only a few symbols, there are likely to be fewer communication errors, i.e. errors arising from modulation/demodulation errors, and/or noise. On the other hand, the fewer, the less information can be transmitted in a given time interval. Conversely, with symbols, information can be transmitted more rapidly. However, for a fixed bandwidth, the frequencies would be closer together. The symbols would thus be harder to distinguish from each other.

In one embodiment, a suitable compromise is achieved by providing a sixteen symbol alphabet, with each symbol being a four-bit word. This particular encoding scheme, referred to as “16-ary FSK,” provides a modulation rate near 125 kilobytes per second. The selection of 16-ary FSK for encoding information onto an RF carrier enables the resulting RF modulated signal to fit into the required 363 kilohertz bandwidth. However, different numbers of symbols can be selected for other applications, or to accommodate improvements in hardware and in noise-reduction methods.

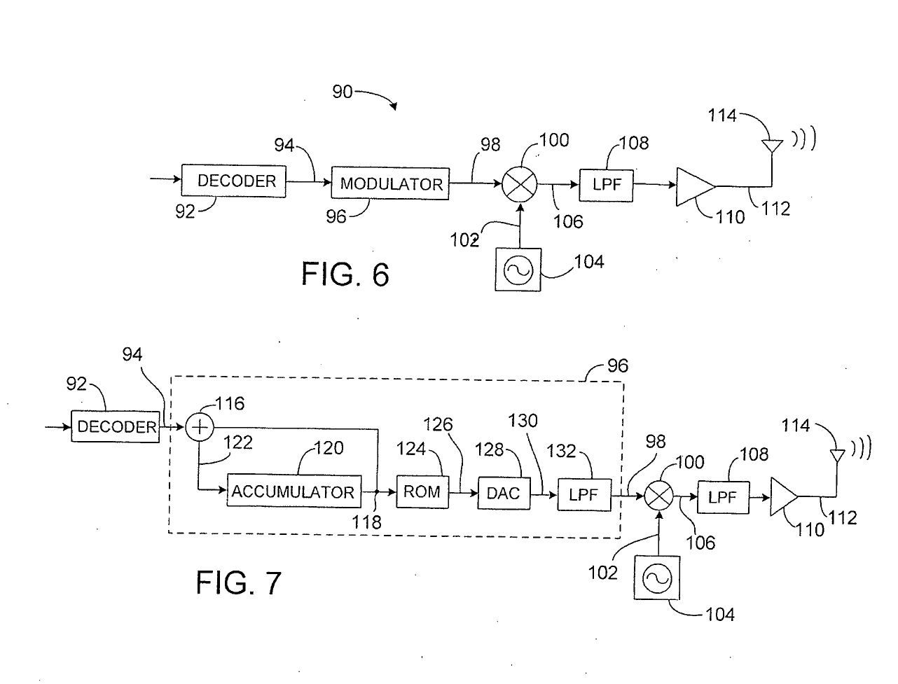

A variety of ways can be used to generate a 16-ary FSK carrier. In one embodiment, shown in FIG. 6, a transmitter 90 includes a decoder 92 for receiving, from the speech processor 16, information representative of ambient sounds. The decoder 92 identifies the particular symbol that is to be transmitted and maps that symbol to the appropriate frequency. The decoder 92 thus provides a selection signal 94 to a modulator 96. The modulator 96 then generates a sensory signal 98 having a frequency that represents the desired symbol.

A mixer 100 receives this sensory signal 98 and combines it with a carrier 102 provided by an oscillator 104. The resulting modulated carrier 106 is filtered by an output low-pass filter 108 and amplified by an amplifier 110. Since the modulated carrier 106 is not severely band-limited, the amplifier 110 can be a class C, D, or even E amplifier. The output of the amplifier 110, which is the telemetry signal 112, is then provided to an antenna 114 to be radiated toward the cochlear implantation portion 12.

One challenge in building the transmitter 90 is that of generating, in real time, a sensory signal having a frequency corresponding to a particular symbol. In one embodiment, shown in FIG. 7, this is carried out in part by direct digital synthesis.

In direct digital synthesis, the decoder 92 provides the selection signal 94 to an adder 116 whose function is to add the selection signal 94 to an accumulated value 118 stored in an accumulator 120, and to store the resulting sum 122 back in the accumulator 120. The accumulated value 118 in the accumulator 120 is then used to sample a complex exponential waveform (referred to herein as the “stored waveform”) stored in a read-only memory 124. The intervals between samples, and hence the sampling frequency, depend on the selection signal 94.

The resulting samples of the stored waveform generate a “sampled waveform” 126 whose frequency can be different from that of the stored waveform. As noted above, the frequency of the sampled waveform depends in part on the sampling frequency, as determined by the selection signal 94. In this way, symbols represented by the selection signal 94 are translated into sampled waveforms 126 of corresponding frequencies.

The resulting sampled waveform 126 is provided to a DAC 128 (digital-to-analog converter) to generate a corresponding analog signal 130. The process of sampling the stored waveform results in a corresponding analog signal 130 having extraneous high-frequency components. These high-frequency components are removed by a low-pass filter 132. The resulting sensory signal 98 is then provided to the mixer 100.

In another embodiment, shown in FIG. 8, the sixteen symbols are modulated onto two waveforms that are in phase quadrature. A transmitter 90 in this case includes first and second DACs 128A, 128B that provide their analog outputs 130A, 130B to respective first and second low pass filters 132A, 132B. At a first mixer 100A, the output of the first low pass filter 132A modulates a first carrier 102A provided by a first oscillator 104A. At a second mixer 100B, the output of the second low pass filter 132B modulates a second carrier 102B provided by a second oscillator 104B. The first and second carriers 102A, 102B are in phase quadrature. Although in FIG. 8 the first and second carriers 102A, 102B are provided by separate first and second oscillators 104A, 104B, one can instead use a single oscillator with two outputs, one of which is connected to a quadrature phase delay, to provide the first and second carrier 102A, 102B.

The outputs of the first and second mixers 100A, 100B are combined at an output adder 134. The resulting modulated carrier 136 is then filtered by an output low pass filter 108 before being amplified by an amplifier 110 and provided to the antenna 114 for radiation. The embodiment shown in FIG. 8 can thus be used to carry out single sideband modulation of the sensory signal onto the carrier.

The cochlear stimulation portion 12 includes a receiver 200, shown in FIG. 9, for receiving the telemetry signal 112 sent by the transmitter 90. The receiver 200 includes a receiving antenna 202 that receives the telemetry signal 112 and provides it to a limiting amplifier 204. The limiting amplifier 204 clips the upper and lower extremities of the received telemetry signal 112 to form a clipped waveform 206 having a variable frequency. These variations in frequency correspond to the received symbols. The resulting clipped telemetry signal 206 is provided to a phase-locked loop 208.

The phase-locked loop 206 includes a voltage-controlled oscillator (not shown) whose output is a waveform having a frequency that matches that of the clipped telemetry signal. This voltage-controlled oscillator is controlled by an oscillator control signal 210. This oscillator control signal 210, which is thus an indicator of the instantaneous frequency of the clipped telemetry signal 112, is the desired output from the phase-locked loop 208. To eliminate undesirable harmonic frequencies, the oscillator control signal 210 from the phase-locked loop 208 is provided to an active low-pass filter 212.

In principle, the resulting filtered oscillator control signal 214 should take one of sixteen values, each one corresponding to a particular one of the sixteen possible symbols. These sixteen values are found within a frequency interval having an upper and lower bound. By dividing this interval into sixteen sub-intervals and observing which sub-interval the value of the filtered oscillator control signal 214 falls into, one can determined the symbol being transmitted. However, before one can divide an interval into sixteen sub-intervals, one must first know the upper and lower bounds of that interval. This information is provided by a min/max detector 216 that receives the filtered oscillator control signal 214 from the low-pass filter 212 and uses it to estimate the upper and lower bounds of the frequency interval.

The filtered oscillator control signal 214 from the low-pass filter 212, together with the estimates from the min/max detector 216, are then provided to a analog-to-analog (“A/D”) converter 218. The output of the A/D converter 218 is a series of four-bit blocks 220, each of which represents a particular one of the sixteen symbols. As the four-bit blocks 220 arrive, they are held in a 4-bit data latch 222 until their values have become stable enough to be useful in a digital symbol. Once this occurs, the data in the latch 222 is provided to a word decoder 224.

In a particular embodiment, data is sampled from the latch 222 at a sampling frequency of 125 kilohertz. A sampling waveform can be provided by a clock 226 as shown. Alternatively, since the filtered oscillator control signal 214 will have an average frequency of 27.12 megahertz, that signal 214 can be used as a basis for generating a sampled waveform.

The word decoder 224 carries out functions common to many digital systems, such as identifying boundaries between frames, and demultiplexing bits within a frame, for example determining which bits belong to control instructions and which belong to data. The output of the decoder 224 is then used to generate signals that control stimulation of the electrodes 50 shown in FIGS. 4 and 5.

Throughout the foregoing discussion, a signal present on a signal line connecting two components is identified by a reference numeral associated with that signal line. Such a signal may be referred to as an “output” signal with reference to one component and an “input” signal with reference to another component. It is understood that the same signal is meant. Thus, for example, in FIG. 8, the signal 130A maybe referred to as the output signal 130A from the first DAC 128A, the input signal 130A to the first low pass filter 132A, the output signal 130A, input signal 130A, or as simply the signal 130A.

The embodiment described herein is particularly adapted for generating a sensory signal indicative of ambient sound. However, the methods and systems described herein can readily be adapted to transmit and receive data representing other sensory stimuli in a way that satisfies constraints on bandwidth. For example, the sensory signal may be indicative of ambient lighting, in which case the signal can be transmitted to a visual prosthesis.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Having described the invention, and a preferred embodiment thereof, what is claimed as new, and secured by Letters Patent is:

1. A system for stimulating a sensory organ, the system comprising:
an external portion for receiving sensory stimuli in real time and wirelessly transmitting the sensory stimuli in real time as a 16-ary FSK signal having a plurality of frequencies, the external portion including a stored waveform, and wherein the external portion generates the 16-ary FSK signal by sampling the stored waveform at sampling rates indicative of the frequencies; and
an internal portion in data communication with the external portion, the internal portion for receiving the 16-ary FSK signal in real time and for causing stimulation of the sensory organ in response to the 16-ary FSK signal.

2-3. (canceled)

4. The system of claim 1, wherein the external portion further comprises
a memory having stored therein samples of a first waveform, and
an accumulator having stored therein information for retrieving selected samples of the first waveform.

5. The system of claim 1, wherein the receiver further comprises a phase-locked loop for generating a control signal representative of a frequency of the FSK signal.

6. The system of claim 1, wherein
the external portion encodes information representative of ambient sound in the 16-ary FSK signal, and
the internal portion causes stimulation of a cochlea on the basis of the encoded information.

7. The system of claim 6, wherein the internal portion further comprises an array of electrodes disposed to stimulate different portions of the cochlea in response to the encoded information.

8. A method for stimulating a sensory organ, the method comprising:
receiving a series of symbols indicative of an ambient stimuli;
decoding the symbols to produce selection signals;
sampling a stored waveform, wherein the frequency at which the stored waveform is sampled depends on the selection signals;
converting the sampled waveform to an analog waveform;
generating an FSK signal having a plurality of frequencies from the analog waveform, wherein each frequency in the FSK signal is indicative of a particular symbol in the series;
wirelessly transmitting the FSK signal transcutaneously to an implanted receiver; and
stimulating the sensory organ in response to the information encoded in the FSK signal.

9. The method of claim 8, wherein generating an FSK signal further comprises generating a 16-ary FSK signal.

10. (canceled)

11. The method of claim 8, wherein stimulating the sensory organ further comprises providing the FSK signal to a phase-locked loop; and encoding information from the FSK signal into a voltage-controlled oscillator control signal generated by the phase-locked loop.

12. The method of claim 8, further comprising selecting the ambient stimuli to be ambient sound, and wherein stimulating the sensory organ comprises stimulating a cochlea.

13. The method of claim 12, wherein stimulating the cochlea comprises selectively exciting electrodes disposed along the cochlea.

14. A cochlear implant system comprising:
an externally-worn portion for receiving audio stimuli in real time and wireless transmitting the audio stimuli in real time as an FSK signal having a plurality of frequencies, the externally-worn portion including a stored waveform, and wherein the FSK signal is generated by sampling the stored waveform at sampling rates indicative of the frequencies;
an implantable portion having a receiver for wireless reception of the FSK signal in real time;
a processor for generating stimulus signals on the basis of the received FSK signal; and
an electrode array responsive to the stimulus signals.

15. The cochlear implant system of claim 14, wherein the transmitter transmits a 16-ary FSK signal.

16. (canceled)

17. The cochlear implant system of claim 14, wherein the transmitter further comprises:
a memory having stored therein samples of a first waveform, and
an accumulator having stored therein information for retrieving selected samples of the first waveform.

18. The cochlear implant system of claim 14, wherein the receiver comprises a phase-locked loop for generating a control signal representative of a frequency of the FSK signal.

19. (canceled)
